(19) United States
(12) Patent Application Publication
(10) Pub. No.: US 2025/0148593 A1
(43) Pub. Date: May 8, 2025

(54) ANALYSIS METHOD FOR OPTIMIZING NUMBER AND POSITION OF SCREWS USED IN LONG BONE FRACTURE FIXATION SURGERY

(71) Applicant: NATIONAL FORMOSA UNIVERSITY, HUWEI TOWNSHIP (TW)

(72) Inventors: Shou-I Chen, HUWEI TOWNSHIP (TW); Yu-Hsu Chen, HUWEI TOWNSHIP (TW); Shau-Huai Fu, HUWEI TOWNSHIP (TW); Chih-Min Yao, HUWEI TOWNSHIP (TW); Wei-Sheng Hong, HUWEI TOWNSHIP (TW); Mu-Kuang He, HUWEI TOWNSHIP (TW); Fan-Jun Xie, HUWEI TOWNSHIP (TW)

(73) Assignee: NATIONAL FORMOSA UNIVERSITY, HUWEI TOWNSHIP, YUNLIN COUNTY (TW)

(21) Appl. No.: 18/926,788

(22) Filed: Oct. 25, 2024

(30) Foreign Application Priority Data
Nov. 7, 2023 (TW) 112142885

(51) Int. Cl. G06T 7/00; G06T 17/00

(52) U.S. Cl. CPC G06T 7/0012; G06T 17/00; G06T 2207/10081

(57) ABSTRACT

An analysis method for optimizing number and position of screws used in long bone (fracture fixation) surgery, including the following steps. Perform a CT scan on a surgical patient to obtain a medical image to build a long bone 3D model. Analyze an X-ray image to obtain a long bone fracture condition and a bone quality condition. Select a bone plate 3D model from the model database. Import the bone plate 3D model into the long bone 3D model and set an initial fixation position of the bone plate 3D model. Instruct the first artificial intelligence to comprehensively analyze the conditions to automatically select alternative solutions in the model database. Then, use a computer-aided analysis system to analyze the stress distribution of the alternative solutions, and a first preferred solution is obtained through simulating analysis, thereby excluding incorrect or ineffective surgical plans to reduce the analysis time and patient wait time and increasing the surgery success rate.

### BACKGROUND OF THE INVENTION

### Technical Field

The present invention relates generally to a method for simulating and evaluating a bone plate fixation surgery of long bone, and more particularly to an efficient analysis method for optimizing the number and position of the screws used for the bone plate fixation surgery based on an artificial intellectual technology and a computer-aided analysis system to improve the precision and efficiency of the analysis.

### Description of Related Art

Long bones are known to be one of the five types of bones in the human body, supporting the daily load of weight and allowing normal body movement. Long bones include the femur, tibia, and fibula in the lower limbs; and the humerus, radius, and ulna in the arms, among others. Common types of human long bone fractures include transverse fracture, oblique fracture, spiral fracture, and so on. The orthopedists have to determine the number of the bone plates and screws for fixation surgeries depending on the location and types of fractures based on their experience. Due to the differences in the type and location of long bone fractures, orthopedists often need to rely on experience to determine how many screws and bone plates are required for bone fixation surgery and which screw hole positions to select. However, from a biomechanical perspective, the 3D structure formed by the screws and the bone plate directly affects the overall rigidity of the fixation system. Using too many screws can indirectly increase the proportion of bone tissue damage, while using too less screws easily leads to screw loosening, resulting in worse overall femoral stability after surgery, which may affect bone fusion. The above-mentioned factor is crucial to define success after long bone fracture fixation surgery. Therefore, if the biomechanical evaluation or preoperative plan could be conducted before the surgery to determine the optimal number of screws and the ideal locking position and locking direction, which could significantly improve the postoperative bone recovery process.

As to the multiple-hole bone plate fixation system of the long bone fracture surgery, both domestic and international research literature have attempted to conduct mechanical analysis of bone plates through computer-aided engineering finite element method (FEM) models, expecting to provide more comprehensive information for clinical surgeons to assess how many screws should be used for fixation and which screw hole positions should be selected. Since there are numerous screw holes on the bone plates and multiple screws are used, the possible combination of screw placements could be various. Analyzing all possible combinations could take several days, which is not practical. Excessively analyzing the data and outcome could affect the preoperative decision-making of the surgeon. Sometimes, “representative surgical fixation combinations” are employed to conduct a qualitative comparison, but it is still impossible to comprehensively analyze all the possible screw placements, which limits the suggestion of optimizing screw numbers and positions and leads to lower accuracy of the analysis. Therefore, the conventional analysis techniques are unable to efficiently obtain the optimal surgical plan.

In light of this, after years of experience in manufacturing, developing, and designing related products, the inventor of this invention has conducted thorough design and careful evaluation, ultimately arriving at this practical invention.

### BRIEF SUMMARY OF THE INVENTION

In view of the above, the primary objective of the present invention is to solve the existing defects of the conventional technique

The present invention provides an analysis method for optimizing the number and position of screws used in long bone fracture fixation surgery, including the following steps. Perform a computed tomography (CT) scan on a surgical patient to obtain a medical image, and establish a long bone 3D model based on the medical image. Perform an X-ray scan on the surgical patient to obtain at least one X-ray image and analyze the X-ray image to obtain a long bone fracture condition and a bone quality condition. Select a bone plate 3D model from a model database. Import the bone plate 3D model into the long bone 3D model, and set an initial fixation position of the bone plate 3D model. Instruct a first artificial intelligence to comprehensively analyze the long bone 3D model, the initial fixation position of the bone plate 3D model, the long bone fracture condition, and the bone quality condition, and automatically select a plurality of alternative solutions in the model database. Analyze a stress distribution of each of the alternative solutions by a computer-aided analysis system, and obtain a first preferred solution after simulating analysis, wherein the first preferred solution includes a number of the screws and a locking position of the screws.

In an embodiment, the method further includes a second artificial intelligence, wherein the second artificial intelligence calculates numerous amounts of real surgical data via a computer-aided analysis method, and then the machine learning is performed on the calculation results to generate a plurality of surgery recommendations in a mechanical analysis database. When the alternative solution selected by the first artificial intelligence is the same as or determined to be similar to one of the surgery recommendations generated by the second artificial intelligence, the second artificial intelligence directly provides the surgery recommendation based on the trained screw stress/strain distribution as the first preferred solution.

In an embodiment, a final surgical plan conducted on the surgical patient, an outcome, and a postoperative tracking record are imported into the mechanical analysis database. Those data are provided to the second artificial intelligence to perform machine learning, thereby expanding the surgery recommendations in the mechanical analysis database and improving the comprehensive analysis ability of the second artificial intelligence.

In an embodiment, each of the alternative solutions in the model database includes a plurality of classification marks; each of the classification marks comprises at least two items, comprising gender, age, height, weight, and job. After the classification mark corresponding to the condition of the surgical patient is inputted, a certain region of the alternative solutions in the model database is selected.

In an embodiment, the locking position of the screws and the number of the screws in both the first preferred solution and the second preferred solution are inputted into the first artificial intelligence to change the initial fixation position of the bone plate 3D model, allowing the first artificial intelligence to conduct comprehensive analysis based on the locking position of the screws, the number of the screws, the long bone 3D model, the long bone fracture condition, and the bone quality condition to reselect a plurality of alternative solutions in the first artificial intelligence. Then the stress distribution of each of the reselected alternative solutions is analyzed by the computer-aided analysis system to obtain a third preferred solution through simulating analysis.

In an embodiment, a loading ability of the long bone 3D model while standing on one leg is simulated through a biomechanical method. The compressive strength and the torsional strength of each of the first preferred solution, the second preferred solution, and the third preferred solution are analyzed to find an optimal configuration of the bone plate 3D model and the screws.

In an embodiment, after different types of the bone plate 3D model and the screws are substituted to the first preferred solution, the second preferred solution, and the third preferred solution, the computer-aided analysis system analyzes to find an optimal type of the bone plate 3D model and the screws

In an embodiment, the model database utilizes a big data method to collect a great amount of the long bone surgical data, which are adapted to provide the first artificial intelligence as the machine learning material and the verification data. The known input and the verification data are comprehensively analyzed through the machine learning algorithm to automatically find regularities, so that the artificial neural network for bone plate fixation surgery and a plurality of alternative solutions are trained and obtained. After corresponding data are inputted into the first artificial intelligence, a proper amount of the alternative solutions could be obtained through an automatic analysis

In an embodiment, the computer-aided analysis technique adopts the finite element method to simulate and analyze a load state of each of the alternative solutions, and further analyze an external deformation and an internal stress at every site of the bone plate 3D model and the screws.

In an embodiment, the X-ray scan is performed on the normal long bone side of the surgical patient to obtain an X-ray image, and the X-ray image is analyzed to obtain a healthy long bone information and a health bone quality information; in a situation of comminuted fracture, the healthy long bone information is used to assist in the establishing of the long bone 3D model, and the healthy bone quality information is used to replace the bone quality condition, thereby improving the accuracy of the simulating analysis.

The primary objective of the present invention is to instruct the first artificial intelligence to comprehensively analyze the long bone 3D model, the initial fixation position of the bone plate 3D model, the long bone fracture condition, and the bone quality condition, automatically select the plurality of the alternative solutions in the model database. Then, the number of the screws and the locking position of the screws of each of the alternative solutions are imported into the real long bone 3D model of the surgical patient to conduct the simulating analysis, numerous incorrect or invalid surgical plans could be excluded, which effectively reduce the time cost on computer-aided analysis. With such design, the calculation taking a few days could be reduced to a few hours, so that the doctor could obtain the analysis results faster, thereby increasing the surgical success rate and reducing the surgical waiting time of the surgical patient.

The secondary objective of the present invention is the second artificial intelligence calculates numerous amounts of real surgical data via the computer-aided analysis method in advance. When the alternative solution selected by the first artificial intelligence is the same as or determined to be similar to one of the surgery recommendations generated by the second artificial intelligence, the second artificial intelligence directly provides the surgery recommendation based on the trained screw stress distribution as the first preferred solution, thereby saving the time cost on the computer-aided analysis system. Additionally, the trained surgery recommendation provided by the second artificial intelligence could be compared with the analyzing result of the computer-aided analysis system, which could enhance the accuracy and efficiency of the surgical plan decision.

Other objectives, advantages, and novel features of this creation will become more apparent from the following detailed description and the accompanying drawings.

### BRIEF DESCRIPTION OF THE SEVERAL VIEWS OF THE DRAWINGS

FIG. 1 is a schematic diagram of the present invention combining a 3D model of a long bone with a 3D model of a bone plate fixation system;

FIG. 2 is a flowchart showing the steps to obtain the first preferred solution in the present invention;

FIG. 3 is a step schematic diagram (1) of obtaining the first preferred solution in the present invention;

FIG. 4 is a step schematic diagram (2) of obtaining the first preferred solution in the present invention;

FIG. 5 is a step schematic diagram of obtaining the second preferred solution based on the first preferred solution in the present invention;

FIG. 6 is a flowchart of the steps to obtain the second preferred solution based on the first preferred solution in the present invention;

FIG. 7 is a step schematic diagram of obtaining the third preferred solution based on the first and second preferred solutions in the present invention; and

FIG. 8 is a schematic diagram of X-ray imaging of a normal long bone side in the present invention.

### DETAILED DESCRIPTION OF THE INVENTION

To provide the esteemed examiner with a comprehensive understanding of the purpose, features, and effects of the present invention, the following detailed description is provided in conjunction with accompanying figures:

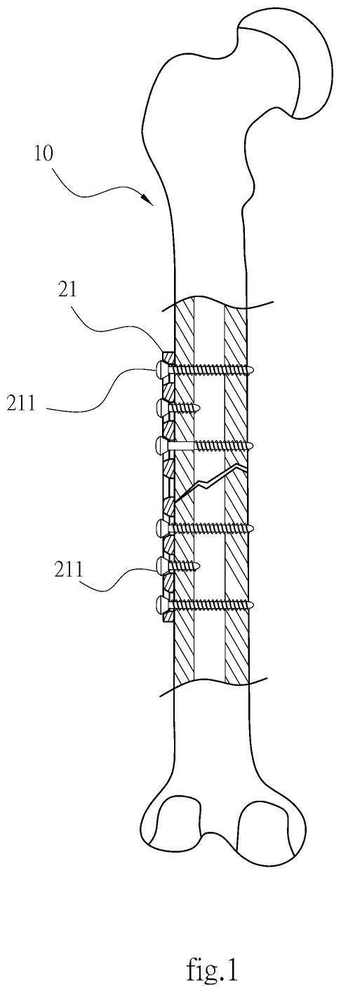

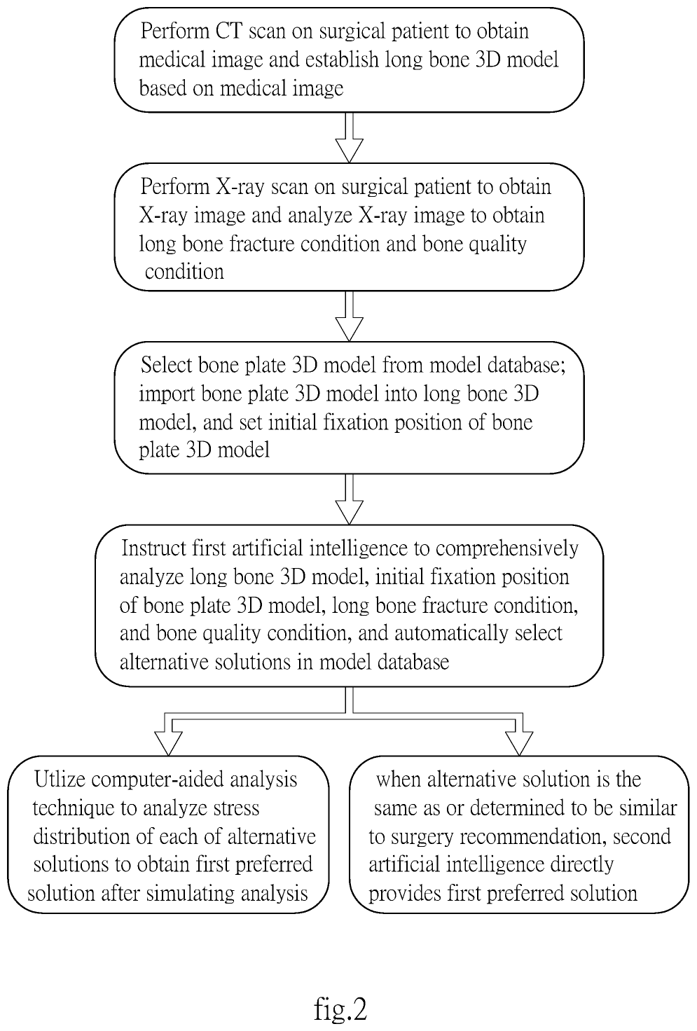

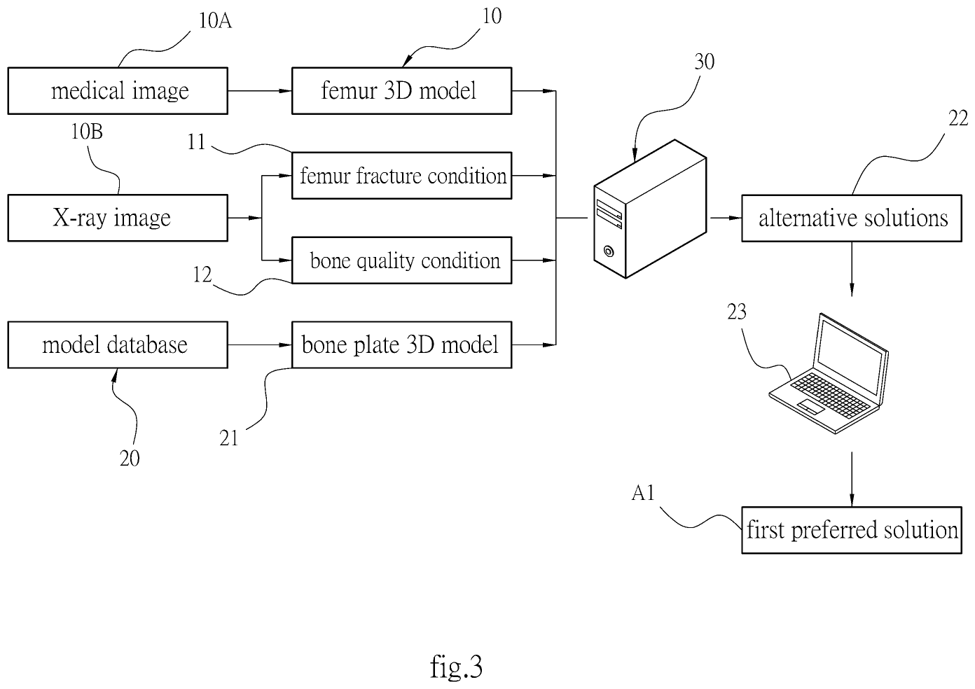

As illustrated in FIG. 1, FIG. 2, and FIG. 3, an efficient analysis method for optimizing the number and position of the screws used for the bone plate fixation surgery includes the following steps:

Step 1: A computed tomography (CT) scan is performed on a surgical patient to obtain at least one medical image 10A, and a long bone 3D model 10 is established based on the medical image 10A. More specifically, the CT images of the surgical patient are obtained by utilizing a CT scanning device. Then, the image processing software is used to capture an outer contour of the long bone from the CT images, and an inner contour of the long bone is captured along the diaphyseal region of the long bone in the CT images. Utilizing the triangular mesh technique to establish an outer surface geometry and an inner surface geometry of the long bone. Finally, data of the outer and inner surface geometry are imported into the computer-aided design (CAD) software to generate the geometric solid surfaces, thereby obtaining the long bone 3D model 10. The term “long bone” refers to elongated bones, such as the femur, tibia, humerus, or ulna.

Step 2: An X-ray scan is performed on the surgical patient to obtain at least one X-ray image 10B, and the X-ray image 10B is analyzed to obtain a long bone fracture condition 11 and a bone quality condition 12, wherein the long bone fracture condition could include a fracture location, a degree of bone fragmentation, and a fracture volume of the long bone. In other embodiments, the long bone fracture condition 11 and the bone quality condition 12 could be obtained by other simpler or more complex methods, and the condition information is not limited to be obtained by the aforementioned method.

Step 3: A bone plate 3D model 21 is selected from a model database 20, and the bone plate 3D model 21 includes a plurality of screws 211, and then the bone plate 3D model 21 is imported into the long bone 3D model 10 and set an initial fixation position of the bone plate 3D model 21, namely the bone plate 3D model 21 and the initial fixation position chosen by a doctor based on the practical experience. The solid model of the bone plate 3D model 21 and components of the screws 211 are pre-established via the drawing software and stored in the model database 20. The aforementioned bone plate 3D model 21 could also be obtained through the big data method from the medical system, namely analyzing the usable information to establish the bone plate 3D model 21. The bone plate 3D models 21 have various shapes, hole numbers, and materials; and the corresponding screws 211 have various diameters, lengths, types, and materials.

Step 4: A first artificial intelligence 30 is instructed to comprehensively analyze the long bone 3D model 10, the initial fixation position of the bone plate 3D model 21, the long bone fracture condition 11, and the bone quality condition 12, and automatically select a plurality of alternative solutions 22 in the model database 20. Some of the aforementioned analyzed data is optional. For example, when the bone quality condition 12 is not input, more amount of the alternative solutions 22 could be obtained. At that time, the first artificial intelligence 30 analyzes and selects the alternative solutions 22, which are relatively feasible, to control the number of the alternative solutions 22, thereby keeping the analysis efficient. The function is achieved by the machine learning of the first artificial intelligence 30. More specifically, the generation of the alternative solutions 22 is inputting the simplified parameter conditions to the first artificial intelligence 30 to generate numerous analyzed data of the numbers and the locking positions of the screws 211, and the analyzed data is verified by the machine learning algorithm, and then the analyzed data is analyzed and classified to allow the first artificial intelligence 30 to identify the differences. Therefore, the stored data could be effectively expanded to become the alternative solutions 22. In order to find the preferred number and locking position of the screws 211, the machine learning algorithm could adopt the optimization method or the iterative method to find the preferred solution. For the optimization method, the condition is inputted into the model database 20 to conduct the analysis and search until the minimum (or maximum) value of the inputted condition is found, so that an appropriate amount of the alternative solutions 22, which is probably adopted, could be found, thereby reducing the calculating work of the computer-aided analysis.

Step 5: The stress distributions of each of the alternative solutions 22 are analyzed by a computer-aided analysis system 23, and a first preferred solution 100 is obtained after simulating analysis, wherein the first preferred solution 100 includes the number of the screws 211 and the locking position of the screws 211, and the doctor could take the first preferred solution 100 as the surgical plan, thereby reducing the medical error rate based on human judgment. In other words, the number and the locking position of the screws 211 in the alternative solutions 22 are imported to the real long bone 3D model 10 of the surgical patient to conduct the simulating analysis, numerous incorrect or invalid surgical plans could be excluded, which effectively reduce the time cost on computer-aided analysis. With such a design, the calculation taking a few days could be reduced to a few hours, so that the doctor could obtain the analysis results faster, thereby increasing the surgical success rate and reducing the surgical waiting time of the surgical patient.

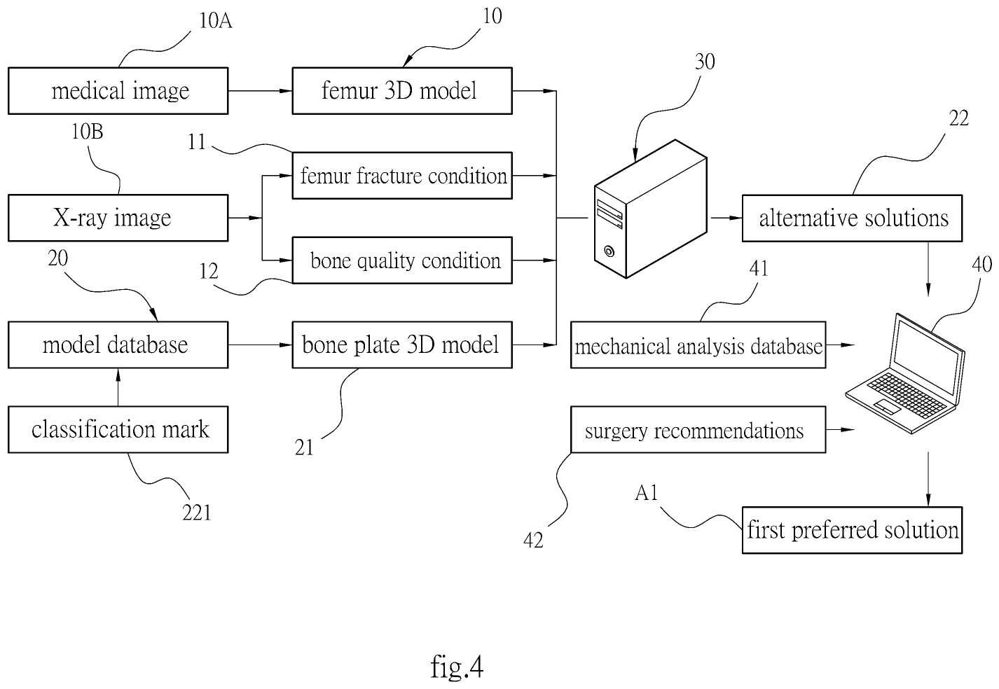

Step 6: As illustrated in FIG. 4, Step 6 could be conducted before Step 5 or simultaneously with Step 5, wherein in Step 6, a second artificial intelligence 40 is included. The second artificial intelligence 40 calculates numerous amounts of real surgical data via the computer-aided analysis method, and then machine learning is performed on the calculation results to generate a plurality of surgery recommendations 42 in a mechanical analysis database 41. When the alternative solution 22 selected by the first artificial intelligence 30 is the same as or determined to be similar to one of the surgery recommendations 42 generated by the second artificial intelligence 40, the second artificial intelligence 40 directly provides the surgery recommendation based on the trained screw stress distribution as the first preferred solution 100, thereby saving the time cost on the computer-aided analysis system 23 in Step 5 and directly generating the first preferred solution 100. In other words, the calculation time in the computer-aided analysis system 23 could be saved. In other embodiments, the trained surgery recommendation provided by the second artificial intelligence 40 could be compared with the analyzed result generated by the computer-aided analysis system 23 in Step 5, thereby increasing the accuracy and efficiency of the surgical plan decision. Furthermore, after the operation, the final surgical plan conducted on the surgical patient, the outcome, and the postoperative tracking record could be imported into the mechanical analysis database 41, which could be provided to the second artificial intelligence 40 to perform machine learning, thereby expanding the surgery recommendations 42 in the mechanical analysis database 41 and improving the comprehensive analysis ability of the second artificial intelligence 40.

As illustrated in FIG. 1 and FIG. 4, each of the alternative solutions 22 in the model database 20 includes a plurality of classification marks 221, wherein each of the classification marks 221 includes at least two of the items, including gender, age, height, weight, and job. After the classification mark 221 corresponding to the condition of the surgical patient is inputted, a certain region of the alternative solutions 22 in the model database 20 is selected. This process could be preparatory work before the selection of the alternative solutions 22 made by the first artificial intelligence 30, thereby enhancing the accuracy of the selection of the alternative solutions 22 made by the first artificial intelligence 30. In case the first artificial intelligence 30 is unable to obtain enough amount of the alternative solutions 22 after the certain regions of the alternative solutions 22 are selected, the first artificial intelligence 30 could reduce the classification mark 221 according to the set criteria to increase the amount of the alternative solutions 22 that are waiting for analysis.

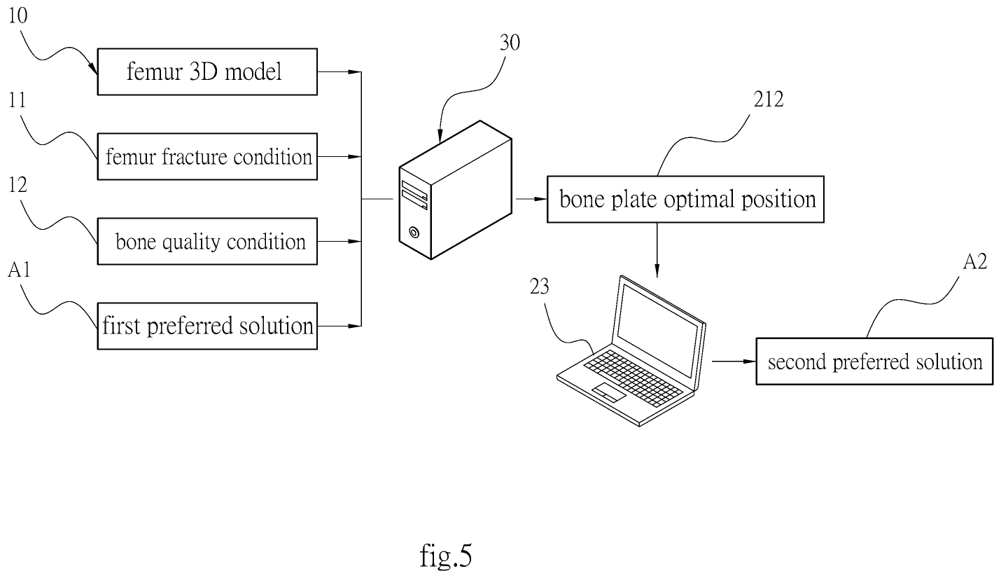

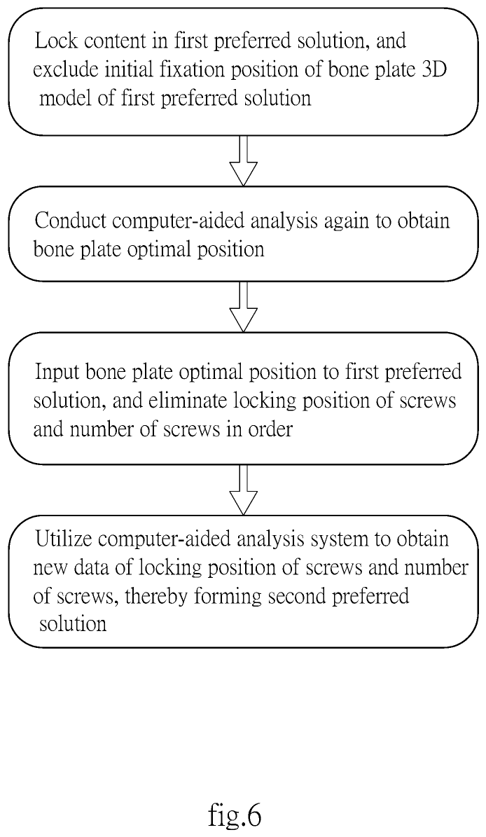

As illustrated in FIG. 1, FIG. 3, FIG. 5, and FIG. 6, all content in the first preferred solution 100 is locked, and the initial fixation position of the bone plate 3D model 21 of the first preferred solution 100 is excluded. After that, the computer-aided analysis system 23 conducts analysis again to obtain a bone plate optimal position 212. Then, the bone plate optimal position 212 is substituted into the first preferred solution 100, and the locking position of the screws 211 and the number of the screws 211 are eliminated in order, and the computer-aided analysis system 23 is utilized to obtain new data of the locking position of the screws 211 and the number of the screws 211, thereby forming a second preferred solution 200. More specifically, the bone plate optimal position 212 is slightly adjusted upward, downward, leftward, and rightward along the initial fixation position of the bone plate 3D model 21. When the bone plate 3D model 21 is slightly adjusted upward and the analyzing result is preferred, the upward adjusted position of the bone plate optimal position 212 is confirmed. After analyzed the adjusted position sequentially, the bone plate optimal position 212 could be obtained. Then, the bone plate optimal position 212 and the number of the screws 211 are analyzed again to obtain new data of the locking position of the screws 211. The bone plate optimal position 212 and the new data of the locking position of the screws 211 are analyzed afterward to obtain a new data of the number of the screws 211. By utilizing the aforementioned steps, the second preferred solution 200 is obtained, and the doctor could choose one of the first preferred solution 100 and the second preferred solution 200 as the surgical plan.

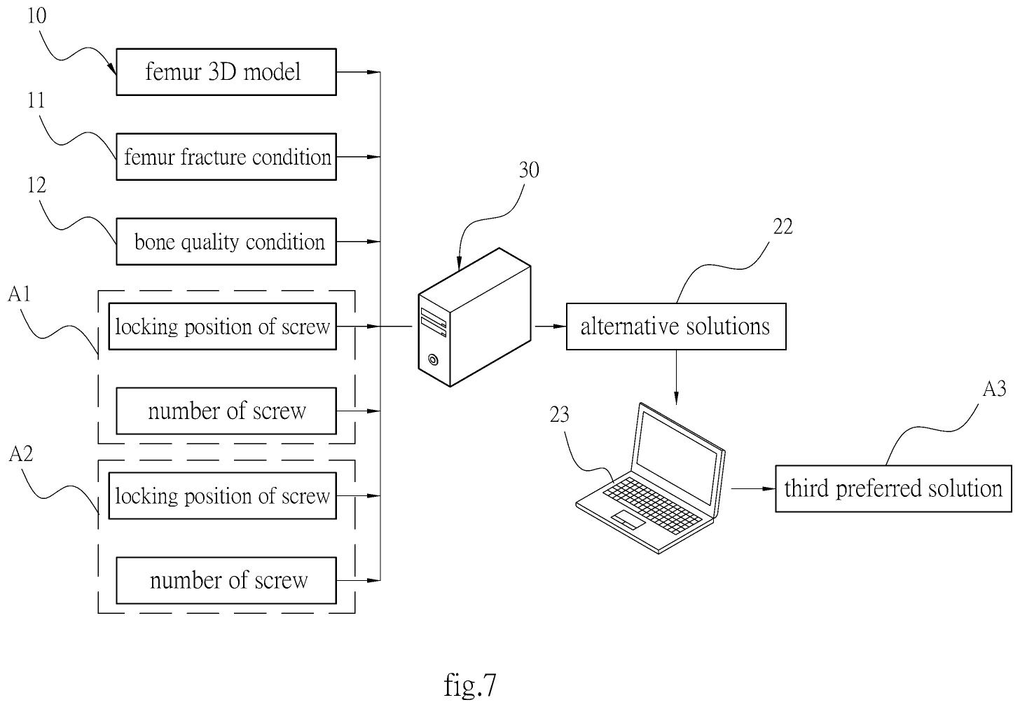

As illustrated in FIG. 1, FIG. 3, FIG. 5, and FIG. 7, the locking position of the screws 211 and the number of the screws 211 of both of the first preferred solution 100 and the second preferred solution 200 are inputted into the first artificial intelligence 30 to change the initial fixation position of the bone plate 3D model 21, allowing the first artificial intelligence 30 to conduct comprehensive analysis based on the locking position of the screws 211, the number of the screws 211, the long bone 3D model 10, the long bone fracture condition 11, and the bone quality condition 12 to reselect a plurality of alternative solutions 22 in the first artificial intelligence 30. After that, the stress distribution of each of the reselected alternative solutions 22 is analyzed by the computer-aided analysis system 23 to obtain a third preferred solution 300 through simulating analysis, thereby preventing the doctor from misjudging the initial fixation position of the bone plate 3D model 21. Likewise, the method allows the doctor to set the initial fixation position ambiguously (exact accuracy is not necessary). At this time, the doctor could choose one of the first preferred solution 100, the second preferred solution 200, and the third preferred solution 300 as the surgical plan.

In an embodiment, simulating the loading ability of the long bone 3D model 10 while standing on one leg through a biomechanical method. Analyze the compressive strength and the torsional strength of each of the first preferred solution 100, the second preferred solution 200, and the third preferred solution 300 to find an optimal configuration of the bone plate 3D model 21 and the screws 211. In an embodiment, different types of the bone plate 3D model 21 and the screws 211 are substituted to the first preferred solution 100, the second preferred solution 200, the third preferred solution 300, and then the computer-aided analysis system 23 conducts an analysis to find an optimal type of the bone plate 3D model 21 and the screws 211, wherein the type includes shape, dimension, material, and so on.

As illustrated in FIG. 1 and FIG. 3, the final surgical plan conducted on the surgical patient, the outcome, and the postoperative tracking record could be imported into the model database 20, and these data are provided for the first artificial intelligence 30 to perform machine learning, thereby expanding the alternative solutions 22 in the model database 20 and improving the comprehensive analysis ability of the first artificial intelligence 40. The model database 20 utilizes a big data method to collect a great amount of the long bone surgical data, which could provide the first artificial intelligence 30 as the machine learning material and the verification data. Through the machine learning algorithm, the known input and the verification data are comprehensively analyzed to automatically find the regularities, and the artificial neural network for bone plate surgery and a plurality of alternative solutions 22 are trained and obtained. Therefore, after the corresponding data are inputted into the first artificial intelligence 30, a proper amount of the alternative solutions 22 could be obtained through an automatic analysis. In the embodiment, the computer-aided analysis system 23 adopts the finite element method to simulate and analyze the load state of each of the alternative solutions 22, and further analyze the external deformation and the internal stress at every site of the bone plate 3D model 21 and the screws 211.

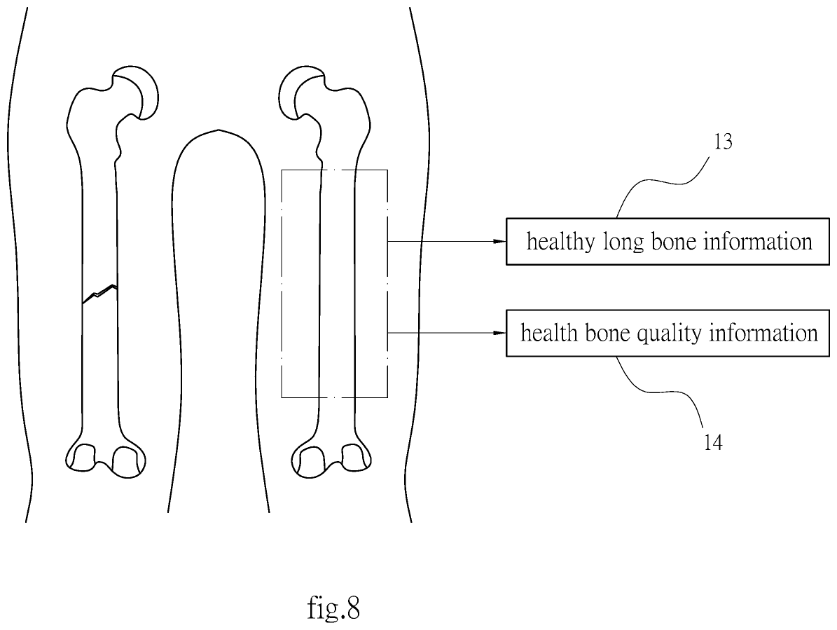

As illustrated in FIG. 1 and FIG. 8, performing the X-ray scan on the normal long bone side of the surgical patient to obtain an X-ray image, and analyze the X-ray image to obtain a healthy long bone information 13 and a health bone quality information 14. In a situation of comminuted fracture, the healthy long bone information 13 is used to assist in establishing of the long bone 3D model 10, and the healthy bone quality information 14 is used to replace the bone quality condition 12, thereby improving the accuracy of the simulating analysis.

It must be pointed out that the embodiment described above is only a preferred embodiment of the present invention. It is understood that the present invention is not limited to these examples and embodiments. All equivalent methods that employ the concepts disclosed in this specification and the appended claims should fall within the scope of the present invention.

What is claimed is:

1. An analysis method for optimizing number and position of screws used in a long bone fracture fixation surgery, comprising:
performing a computed tomography (CT) scan on a surgical patient to obtain at least one medical image and establishing a long bone 3D model based on the medical image;
performing an X-ray scan on the surgical patient to obtain at least one X-ray image and analyzing the X-ray image to obtain a long bone fracture condition and a bone quality condition;
selecting a bone plate 3D model from a model database; importing the bone plate 3D model into the long bone 3D model, and setting an initial fixation position of the bone plate 3D model;
instructing a first artificial intelligence to comprehensively analyze the long bone 3D model, the initial fixation position of the bone plate 3D model, the long bone fracture condition, and the bone quality condition, and automatically selecting a plurality of alternative solutions in the model database;
analyzing a stress distribution of each of the alternative solutions by a computer-aided analysis system, and obtaining a first preferred solution after simulating analysis, wherein the first preferred solution comprises a number of the screws and a locking position of the screws.

2. The analysis method as claimed in claim 1, further comprising a second artificial intelligence, wherein the second artificial intelligence calculates numerous amounts of real surgical data via a computer-aided analysis method, and then the machine learning is performed on the calculation results to generate a plurality of surgery recommendations in a mechanical analysis database; when the alternative solution selected by the first artificial intelligence is the same as or determined to be similar to one of the surgery recommendations generated by the second artificial intelligence, the second artificial intelligence directly provides the surgery recommendation based on the trained screw stress/strain distribution as the first preferred solution.

3. The analysis method as claimed in claim 2, wherein a final surgical plan conducted on the surgical patient, an outcome, and a postoperative tracking record are imported into the mechanical analysis database, which are provided to the second artificial intelligence to perform machine learning, thereby expanding the surgery recommendations in the mechanical analysis database and improving the comprehensive analysis ability of the second artificial intelligence.

4. The analysis method as claimed in claim 1, wherein each of the alternative solutions in the model database comprises a plurality of classification marks; each of the classification marks comprises at least two items, comprising gender, age, height, weight, and job; after the classification mark corresponding to the condition of the surgical patient is inputted, a certain region of the alternative solutions in the model database is selected.

5. The analysis method as claimed in claim 1, wherein content in the first preferred solution is locked, and the initial fixation position of the bone plate 3D model of the first preferred solution is excluded; the computer-aided analysis system conducts analysis again to obtain a bone plate optimal position; then, the bone plate optimal position is substituted into the first preferred solution, and the locking position of the screws and the number of the screws are eliminated in order, and the computer-aided analysis system is utilized to obtain new data of the locking position of the screws and the number of the screws, thereby forming a second preferred solution.

6. The analysis method as claimed in claim 5, wherein the locking position of the screws and the number of the screws in both the first preferred solution and the second preferred solution are inputted into the first artificial intelligence to change the initial fixation position of the bone plate 3D model, allowing the first artificial intelligence to conduct comprehensive analysis based on the locking position of the screws, the number of the screws, the long bone 3D model, the long bone fracture condition, and the bone quality condition to reselect a plurality of alternative solutions in the first artificial intelligence; then, the stress distribution of each of the reselected alternative solutions is analyzed by the computer-aided analysis system to obtain a third preferred solution through simulating analysis.

7. The analysis method as claimed in claim 6, wherein a loading ability of the long bone 3D model while standing on one leg is simulated through a biomechanical method; the compressive strength and the torsional strength of each of the first preferred solution, the second preferred solution, and the third preferred solution are analyzed to find an optimal configuration of the bone plate 3D model and the screws.

8. The analysis method as claimed in claim 6, wherein after different types of the bone plate 3D model and the screws are substituted to the first preferred solution, the second preferred solution, and the third preferred solution, the computer-aided analysis system analyzes to find an optimal type of the bone plate 3D model and the screws.

9. The analysis method as claimed in claim 8, wherein the model database utilizes a big data method to collect a great amount of the long bone surgical data, which are adapted to provide the first artificial intelligence as the machine learning material and the verification data; the known input and the verification data are comprehensively analyzed through the machine learning algorithm to automatically find regularities, so that the artificial neural network for bone plate fixation surgery and a plurality of alternative solutions are trained and obtained; after corresponding data are inputted into the first artificial intelligence, a proper amount of the alternative solutions could be obtained through an automatic analysis.

10. The analysis method as claimed in claim 1, wherein the computer-aided analysis technique adopts the finite element method to simulate and analyze a load state of each of the alternative solutions, and further analyze an external deformation and an internal stress at every site of the bone plate 3D model and the screws.

11. The analysis method as claimed in claim 1, wherein the X-ray scan is performed on the normal long bone side of the surgical patient to obtain an X-ray image, and the X-ray image is analyzed to obtain a healthy long bone information and a health bone quality information; in a situation of comminuted fracture, the healthy long bone information is used to assist in the establishing of the long bone 3D model, and the healthy bone quality information is used to replace the bone quality condition, thereby improving the accuracy of the simulating analysis.
